(19) United States
(12) Patent Application Publication
(10) Pub. No.: US 2017/0325289 A1
(43) Pub. Date: Nov. 9, 2017

(54) ELECTRONIC CIGARETTE AND ATOMIZATION CONTROL METHOD THEREOF

(71) Applicant: HUIZHOU KIMREE TECHNOLOGY CO., LTD., Huizhou, Guangdong (CN)

(72) Inventor: Qiuming Liu, Shenzhen, Guangdong (CN)

(73) Assignee: Huizhou Kimree Technology Co., Ltd., Huizhou, Guangdong (CN)

(21) Appl. No.: 15/527,009

(22) PCT Filed: Nov. 14, 2014
(86) PCT No.: PCT/CN2014/091156

§ 371 Date: May 15, 2017

(51) Int. Cl. H05B 3/00; A61M 15/06; H05B 1/02; A24F 47/00; A61M 16/00; H02J 7/00; A61M 11/04

(52) U.S. Cl. CPC H05B 3/0014; A24F 47/008; A61M 15/06; H05B 1/0216; A61M 11/042; A61M 2016/0039; H02J 2007/0001; H02J 2007/0095; H05B 1/0211; A61M 2205/8206

(57) ABSTRACT

The present invention is disclosed an electronic cigarette and an atomization control method thereof. The electronic cigarette comprises an atomization assembly, a battery assembly, a control module electrically connected to the power source in the battery assembly, a heating wire assembly in the atomization assembly, and an airflow sensor for transmitting a smoking signal to the control module when it is detected that a user is smoking. The present invention has the beneficial effects that the electronic cigarette is actuated after the airflow sensor is triggered when conductors arranged at different positions are in contact with the human skin, so as to reduce probability of false triggering of the electronic cigarette; and during use, this structure conforms to the users' habit of smoking by holding the battery assembly in their fingers and holding the suction nozzle in their mouths, which is convenient for the user and improves the user experience.

### FIELD OF THE INVENTION

The present application relates to a technical field of an electronic cigarette, and more particularly relates to an electronic cigarette and an atomization control method thereof.

### BACKGROUND OF THE INVENTION

In the prior art, electronic cigarettes generally comprise two parts which are an atomization assembly for atomizing e-liquid and a battery assembly for supplying power to the atomization assembly. Among them, an existing electronic cigarette integrated an airflow sensor with a control circuit to control the electronic cigarette, when the user smoking electronic cigarette, the electronic cigarette air pressure will change, resulting in deformation of a thin film capacitor of the airflow sensor. When the amount of the deformation of the thin film capacitor reaches a threshold, a trigger signal is sent to the microprocessor in the control circuit to ensure the microprocessor to control the battery assembly to supply power to the atomization assembly to atomize e-liquid.

However, such an electronic cigarette in a packaging or transport process, is prone to cause a change of the air pressure in the electronic cigarette, which causes the electronic cigarette to work automatically. The electronic cigarette automatically working for a long time is likely to cause a risk of fire and explosion, while the electronic cigarette automatically working for a long time leads to a problem that deterioration and taste of the atomized e-liquid are changed after the atomized e-liquid is repeatedly oxidized by the air.

Another existing electronic cigarette is to replace the airflow sensor through a mechanical key switch to control the electronic cigarette to achieve an atomization of the liquid. Because a frequency of pressing the key switch is high when smoking the electronic cigarette, the key switch is likely to fail during a long pressing process, then some electronic cigarettes are controlled by using touch switches instead of the above key switches. However, a touch switch is usually provided at a certain position on an outer peripheral surface of the battery assembly, and as long as the touch switch is touched, the electronic cigarette is operated, it is easy to touch the touch switch when packaging or holding the electronic cigarette, thereby leading to an automatic work situation of the electronic cigarette.

### SUMMARY OF THE INVENTION

Technical problems to be solved in the present invention is to provide an electronic cigarette and an atomization control method thereof aiming at defects in the prior art.

A technical proposal for solving the technical problem of the invention is shown as following.

Provide an electronic cigarette comprising an atomization assembly and a battery assembly, a heating wire assembly is defined in the atomization assembly, the battery assembly is provided with a power source for supplying electrical power to the heating wire assembly so that the atomization assembly atomizes e-liquid to form smoke; the electronic cigarette further comprises a control module electrically connected to the power source and the heating wire assembly, respectively, and an airflow sensor configured for transmitting a smoking signal to the control module when the airflow sensor detects that a user is smoking; a first conductor is provided on an outer peripheral surface of the battery assembly; a suction nozzle for a user to smoke is provided on an end of the atomization assembly opposite to the battery assembly; and a second conductor is provided on an outer peripheral surface of the suction nozzle; one conductive sleeve of the first conductor and the second conductor is electrically connected to the control module and the other conductor is electrically connected to the power source;

The first conductor and the second conductor are contacted and then conducted by the user's skin while the user is smoking, the control module is configured for controlling the power source to supply the electrical power to the heating wire assembly to atomize the e-liquid when the control module detects the smoking signal and a conduction signal generated by that the first conductor and the second conductor are conducted.

In the electronic cigarette according to the present invention, the atomization assembly comprises an e-liquid cup assembly, the e-liquid cup assembly comprises an e-liquid reservoir and a vent pipe, the vent pipe is inserted in the e-liquid reservoir and configured for discharging smoke atomized by the atomization assembly from the suction nozzle, one end of the e-liquid cup assembly is provided with the suction nozzle made of a conductive material, an outer surface of the suction nozzle is configured as the second conductor, the other end of the e-liquid cup assembly is provided with an atomization core for atomizing the e-liquid, at least one end of the atomization core is inserted into the e-liquid cup assembly.

One end of the atomization core is connected to the vent pipe, the other end of the atomization core is connected with the battery assembly, the control module is defined in the battery assembly, two ends of the vent pipe are electrically connected to the suction nozzle and the atomization core, respectively, the suction nozzle is electrically connected to the control module via the vent pipe and the atomization core.

In the electronic cigarette according to the present invention, a smoke passage and an elastic conductive arm are defined in the atomization core, the smoke passage is communicated with the vent pipe, the elastic conductive arm extends into the smoke passage are defined in the atomization core, the elastic conductive arm is electrically connected to the control module, the vent pipe is inserted into the smoke passage, the elastic conductive arm is abutted against a side wall of the vent pipe.

In the electronic cigarette according to the present invention, the atomization core further comprises a hollow atomization seat; the smoke passage is defined in the atomization seat, a side wall of the atomization seat is provided with an e-liquid entering channel communicated with an e-liquid storage cavity in the e-liquid cup assembly, the heating wire assembly is defined on the smoke passage; an end of the atomization seat is connected with an atomization electrode assembly configured for being electrically connected to the battery assembly.

In the electronic cigarette according to the present invention, the battery assembly comprises the first conductive sleeve configured as the first conductor, the first conductive sleeve receives a battery configured as the power source, one end of the first conductive sleeve is provided with a first connector for being detachably connected the atomization assembly, the other end of the first conductive sleeve is provided with an end cap configured for covering the first conductive sleeve.

In the electronic cigarette according to the present invention, the battery assembly comprises a battery case, the first conductor is a first conductive sleeve made of a conductive paper, the first conductive sleeve is sleeved on the battery case and covers an entire outer peripheral surface of the battery case, the battery case receives a battery configured as the power source, one end of the battery case is provided with a first connector for being detachably connected the atomization assembly, the other end of the battery case is provided with an end cap configured for covering the first conductive sleeve.

In the electronic cigarette according to the present invention, an outer peripheral surface of the atomization assembly is provided with an insulating paper sleeve defined coaxially with the first conductor, the insulating paper sleeve is located between the first conductor sleeve and the second conductor.

In the electronic cigarette according to the present invention, the battery assembly and the atomization assembly are detachably connected, and the detachable connection between the battery assembly and the atomization assembly is a threaded connection structure or an interlocking connection structure.

In the electronic cigarette according to the present invention, the control module comprises a conductive sleeve conduction signal detection circuit, a microprocessor, a transistor switch unit, the transistor switch unit is electrically connected to the microprocessor and the heating wire assembly;

The conductive sleeve conduction signal detection circuit is configured for detecting whether or not the first conductive sleeve and the second conductor are conducted, and transmitting a trigger signal generated after the conduction to the microprocessor, the microprocessor is configured for controlling an on/off state of the transistor switch unit in accordance with the trigger signal and the smoking signal which is outputted by the airflow sensor to control whether the atomization assembly atomizes the e-liquid.

In the electronic cigarette according to the present invention, the conductive sleeve conduction signal detection circuit comprises a filter circuit electrically connected to the second conductor for filtering an inputted conduction signal, and a transistor switching circuit electrically connected an output end of the filter circuit, the transistor switching circuit is configured to receive the conduction signal after filtering and output a switching signal to the microprocessor.

In the electronic cigarette according to the present invention, the filter circuit comprises a first resistor, a second resistor, a first capacitor and a second capacitor, a common terminal of the first resistor, the first capacitor and the second capacitor is grounded, the other end of the first resistor, the other end of the first capacitor and one end of the second resistor are electrically connected to the second conductor, the other end of the second resistor and the other end of the second capacitor are connected to the transistor switching circuit.

In the electronic cigarette according to the present invention, the transistor switching circuit comprises a first transistor, a second transistor, a third resistor, a fourth resistor and a fifth resistor; a base of the first transistor is connected with the filter circuit, an emitter of the first transistor is grounded, a collector of the first transistor is connected to one end of the third resistor and one end of the fourth resistor, the other end of the third resistor is electrically connected to the power source, and the other end of the fourth resistor is connected to a base of the second transistor, an emitter of the second resistor is electrically connected to the power source, a collector of the second resistor is electrically connected to one end of the fifth resistor and the microprocessor, and the other end of the fifth resistor is grounded.

In the electronic cigarette according to the present invention, the conductive sleeve conduction signal detection circuit comprises a filter circuit electrically connected to the second conductor for filtering an inputted conduction signal, and a comparison circuit electrically connected to an output terminal of the filter circuit, a positive input terminal of the comparison circuit receives the conduction signal after filtering and outputs a comparison result signal to the microprocessor.

In the electronic cigarette according to the present invention, the battery assembly is provided with a battery configured as the power source; one conductive sleeve selected from both the first conductive sleeve and the second conductor is electrically connected to a positive electrode of the battery.

In the electronic cigarette according to the present invention, the atomization assembly and the battery assembly are arranged coaxially.

The present invention further provides an atomization control method of an electronic cigarette, the electronic cigarette comprises an atomization assembly and a battery assembly, a heating wire assembly is defined in the atomization assembly, the battery assembly is provided with a power source for supplying electrical power to the heating wire assembly so that the atomization assembly atomizes e-liquid to form smoke; the electronic cigarette further comprises a control module electrically connected to the power source and the heating wire assembly respectively, and an airflow sensor configured for transmitting a smoking signal to the control module when the airflow sensor detects that a user is smoking; a first conductor is provided on an outer peripheral surface of the battery assembly; a suction nozzle for a user to smoke is provided on an end of the atomization assembly opposite to the battery assembly; and a second conductor is provided on the outer peripheral surface of the suction nozzle; the control method comprises following steps.

S1. conducting the first conductor and the second conductor with user's skin when the user contacts the first conductor and the second conductor with the skin simultaneously, so as to generate a conduction signal to the control module;

S2. controlling the power source to supply the electrical power to the heating wire assembly to atomize e-liquid when the control module receives the conduction signal and a smoking signal, the conduction signal is generated by that the first conductor and the second conductor are conducted.

Generally, applications of the electronic cigarette and the atomization control method thereof of the present invention have following advantages, the first conductor is provided on the outer peripheral surface of the battery assembly, and the outer peripheral surface of the suction nozzle is provided with a second conductor, when smoking, the user directly holds on the first conductor by fingers and holds on the suction nozzle by a mouth, so as to realize contacts between the conductive sleeves at different positions on the outer surface of the electronic cigarette and the user's skin at the same time, and to transmit the conduction signal to the control module, meanwhile the airflow sensor is triggered, then the airflow sensor outputs a smoking signal to the control module, the control module controls the power source to supply the heating wire assembly to atomize the e-liquid and start the electronic cigarette working after receiving the conduction signal and the smoking signal.

Therefore, the present invention can reduce a probability that the electronic cigarette is mistakenly triggered, effectively solves the problem in the prior art that the electronic cigarette works when the key switch, the touch switch or the airflow sensor is often mistakenly triggered, resulting in a change of deterioration of taste of the e-liquid of the electronic cigarette caused by repeated oxidization, a short electronic cigarette life, low safety and other issues. Moreover, by providing the structure of the present invention, it is possible to reduce conditions of the packaging apparatus for transporting the electronic cigarette, thus to facilitate the transportation and the transportation is more secure. In addition, during a using process, the structure meets user habits that the user can smoke when the fingers are held on the battery assembly and the mouth is held on the suction nozzle, it is more user-friendly, improves user experience, so as to avoid a complex using problem that the touch keys or switches are needed to be touched in the prior art.

### BRIEF DESCRIPTION OF THE DRAWINGS

The present invention will be further described with reference to the accompanying drawings and embodiments in the following.

FIG. 1 is an explosion view of an electronic cigarette provided by one of the preferred embodiments of the present invention;

FIG. 2 is a cross-sectional view of the electronic cigarette shown in FIG. 1;

FIG. 3 is a structural schematic view of the battery assembly of the electronic cigarette shown in FIG. 1;

FIG. 4 is a structural schematic view of the atomization core of the atomization assembly in the electronic cigarette shown in FIG. 1;

FIG. 5 is an explosion view of the atomization core in FIG. 4;

FIG. 6 is a schematic view of the structure of the fixing block of the atomization core shown in FIG. 5;

FIG. 7 is a structural schematic view of the electronic cigarette provided by a second preferred embodiment of the present invention;

FIG. 8 is a schematic view of the structure of the fixing block of the atomization core shown in FIG. 7;

FIG. 9 is a structural diagram of the control module of the electronic cigarette shown in FIG. 1;

FIG. 10 is a configuration diagram of the conductive sleeve conduction signal detection circuit of a first kind of the control module shown in FIG. 9;

FIG. 11 is a configuration diagram of the conductive sleeve conduction signal detection circuit of a second kind of the control module shown in FIG. 9;

FIG. 12 is a circuit diagram of a first kind of the control module shown in FIG. 9;

FIG. 13 is a circuit diagram of a second kind of the control module shown in FIG. 9.

### DETAILED DESCRIPTION OF THE PREFERRED EMBODIMENT

To make the technical feature, objective and effect of the present application be understood more clearly, now the specific implementation of the present application is described in detail with reference to the accompanying drawings and embodiments.

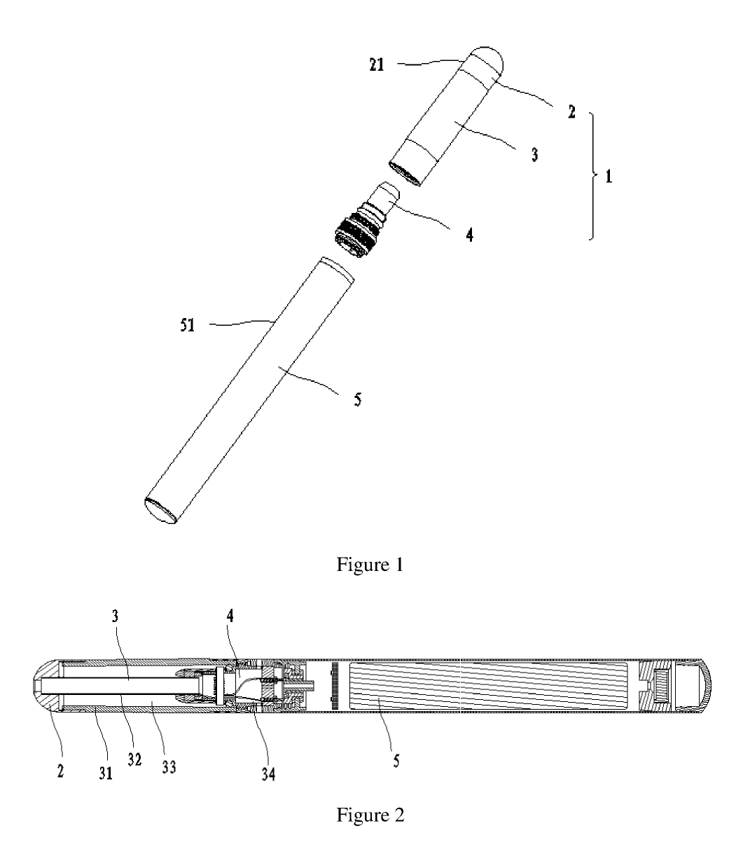

FIG. 1 and FIG. 2 show an electronic cigarette provided by one of the preferred embodiments of the present invention, the electronic cigarette comprises an atomization assembly 1 and a battery assembly 5, the battery assembly 5 is electrically connected to the atomization assembly 1 and is configured for supplying electrical power to the atomization assembly 1, the atomization assembly 1 and the battery assembly 5 are co-axially defined. The atomization assembly 1 comprises a suction nozzle 2, an e-liquid cup assembly 3 and a atomization core 4 which are connected successively, the suction nozzle 2 and the atomization core 3 are respectively connected to two ends of the e-liquid cup assembly 3, and the battery assembly 5 is connected to one end of the atomization core 4 opposite to the suction nozzle 2.

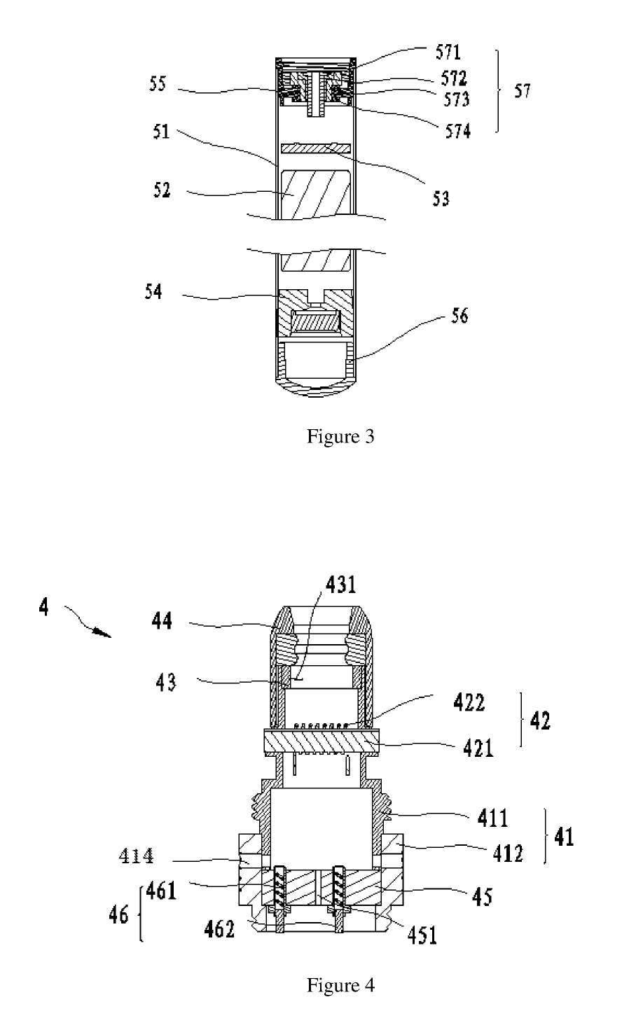

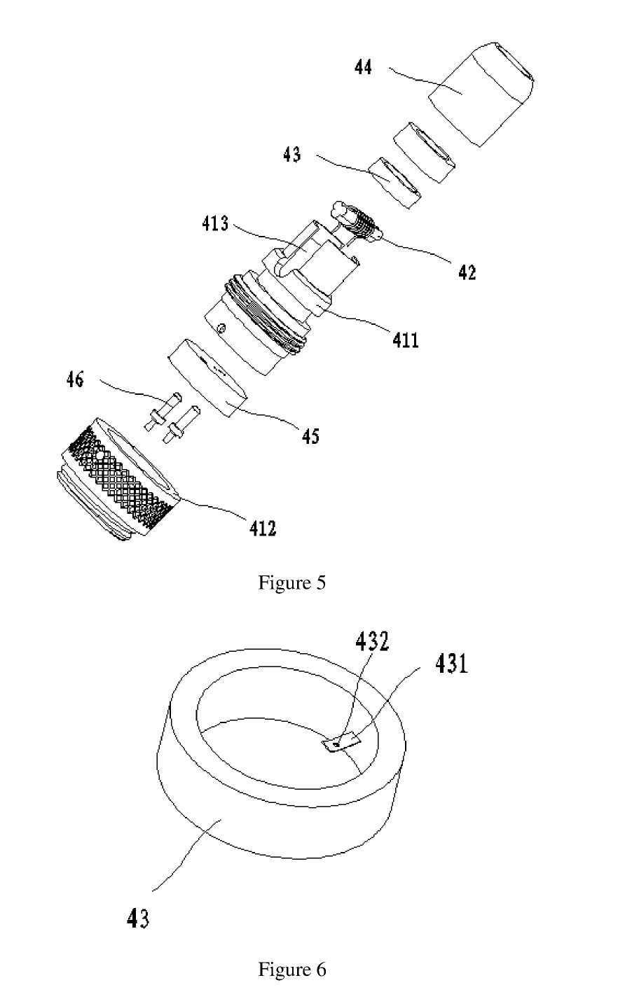

As shown in FIGS. 3-4, a heating wire assembly 42 is defined in the atomization assembly 1, the battery assembly 5 is provided with a power source for supplying electrical power to the heating wire assembly 42 so that the atomization assembly 1 atomizes e-liquid to form smoke. The electronic cigarette further comprises a control module 53 electrically connected to the power source and the heating wire assembly 42, respectively, and an airflow sensor 54 configured for transmitting a smoking signal to the control module 53 when the airflow sensor 54 detects that a user is smoking.

As shown in FIG. 1, a first conductor 51 is provided on an outer peripheral surface of the battery assembly 5, a suction nozzle 2 for a user to smoke with is provided on an end of the atomization assembly 1 opposite to the battery assembly 5; and a second conductor 21 is provided on an outer peripheral surface of the suction nozzle 2; one conductor of the first conductor 51 and the second conductor 21 is electrically connected to the control module 53 and the other conductor is electrically connected to the power source, so that an electrical potential of the conductor electrically connected to the power source is higher than an electrical potential of the conductor electrically connected to the control module 53. Therefore, the conductor electrically connected to the power source can be directly or indirectly electrically connected to the power source, as long as the potential of the conductor is higher than the potential of the conductor electrically connected to the control module 53.

When the user is smoking, the user contacts the first conductive sleeve and the second conductor 21 through skin, respectively, so that the first conductive sleeve and the second conductor 21 are conducted, the control module 53 controls the power source to supply the electrical power to the heating wire assembly 42 to atomize the e-liquid when the control module 53 receives a conduction signal and a smoking signal, the conduction signal is generated by that the first conductive sleeve and the second conductor 21 are conducted.

In the present embodiment, the second conductor 21 is provided at the suction nozzle 2 of the electronic cigarette, the first conductor 51 is provided on the outer peripheral surface of the battery assembly 5, namely, only when the user skin contacts with the first conductor 51 and the second conductor 21, the first conductor 51 and the second conductor 21 are turned on, meanwhile, the airflow sensor 54 outputs a signal, then the electronic cigarette is triggered to atomize, the electronic cigarette operates normally, effectively avoiding a defect of an automatically started electronic cigarette in packages during a process of transporting the electronic cigarette.

As shown in FIG. 1, the first conductor 51 and the second conductor 21 are spaced apart from each other on an outer peripheral wall of the electronic cigarette, and the first conductor 51 is located on the outer peripheral wall of the battery assembly 5, the second conductor 21 is defined on the outer surface of the suction nozzle 2.

Preferably, the first conductor 51 and the second conductor 21 are made of a metal conductive material, so that the structure of the electronic cigarette is reliable. The metal material may be a material such as gold, silver, copper, iron or stainless steel, and is not particularly limited thereto.

As shown in FIG. 3, the battery assembly 5 comprises the first conductor 51, the first conductor 51 is a hollow cylindrical structure, and receives a battery 52 configured as the power source, one end of the first conductor 51 is provided with a connector 55 for being detachably connected the atomization core 4, the other end of the first conductor 51 is provided with an end cap 56 configured for covering the first conductive sleeve. Since the user normally holds on the battery assembly 5 by fingers at the smoking time, the structure is not only simple and compact, but also allows the user to well contact with the first conductor 51 during using, and it prevents the first conductor 51 defined at one point of the battery assembly 5 from being difficultly to be contacted with.

Understandably, in other embodiments, the battery assembly 5 comprises a battery case (not labeled in drawings), the first conductor 51 is a first conductive sleeve made of a conductive paper (not shown in drawings), the first conductive sleeve is sleeved on the battery case and covers an entire outer peripheral surface of the battery case, and the battery case receives a battery 52 configured as the power source, one end of the first conductive sleeve is provided with a connector 55 for being detachably connected the atomization core 4, the other end of the first conductive sleeve is provided with an end cap 56 configured for covering the first conductive sleeve. The present embodiment does not limit a specific structure of the first conductor 51 as long as it can be provided on the outer peripheral surface of the battery assembly 5 and is electrically connected to the control module 53 or the battery 52 of the electronic cigarette. The conductive paper can not only imitate a real cigarette, but also improve the user's comfort.

As shown in FIG. 2, the atomization assembly 1 comprises the suction nozzle 2, the e-liquid assembly 3 and the atomization core 4, the suction nozzle 2 and the atomization core 4 are defined at two ends of the e-liquid assembly 3, respectively. The second conductor 21 is sleeved on the outer peripheral surface of the suction nozzle 2. By providing the second conductor 21 on the suction nozzle 2, it is possible to make it easy for the user to ensure that the second conductor 21 comes into contact with the user's skin when the user is smoking, and then it is more convenient for the user to use and thereby improves the user's experience.

It is preferred that the second conductor 21 is coated on an entire outer peripheral surface of the suction nozzle 2.

Preferably, the second conductor 21 is spaced apart from the first conductor 51 in the battery assembly 5 to ensure an electrical insulation between the first conductor 51 and the second conductor 21. In the present embodiment, the first conductor 51 and the second conductor 21 are respectively provided at two ends of the e-liquid cup assembly 3, and the outer peripheral surface of the e-liquid cup assembly 3 is made of an insulating material to achieve an electrical isolation between the first conductor 51 and the second conductor 21.

Preferably, an outer peripheral surface of the e-liquid cup assembly 3 is provided with an insulating paper sleeve (not shown in drawings) defined coaxially with the first conductor 51 and the second conductor 21, the insulating paper sleeve is located between the first conductor 51 and the second conductor 21.

When the user smokes, the skin comes into contact with the first conductor 51 and the second conductor 21 so that the first conductor 51 and the second conductor 21 are conducted, and the control module 53 controls the power supply to supply the electrical power to the heating wire assembly 42 to atomize the e-liquid to enable the electronic cigarette to operate normally after receiving the conduction signal and the smoking signal, the conduction signal is generated by that the first conductor 51 and the second conductive are conducted.

Specific structures of the e-liquid cup assembly 3 and the suction nozzle 2 of the atomization assembly 1 will be described in detail with reference to the embodiments shown in FIGS. 1-2.

As shown in FIG. 2, the atomization assembly 1 comprises an e-liquid reservoir 31, a vent pipe 32, an e-liquid storage cavity 33 and a connecting member 34.

The e-liquid reservoir 31 has a substantially cylindrical structure, and the atomization core 4 and the suction nozzle 2 are respectively connected to two ends of the e-liquid reservoir 31, the vent pipe 32 is provided in the e-liquid reservoir 31 in an axial direction of the e-liquid reservoir 31, and the e-liquid storage cavity 33 is formed between the e-liquid reservoir 31 and the vent pipe 32, and the e-liquid for being atomized by the atomization core 4 is received in the e-liquid storage cavity 33. In order to facilitate the user to view the remaining amount of the e-liquid in the e-liquid storage cavity 33, in the present embodiment, the e-liquid reservoir 31 is made of a light-permeable material.

The vent pipe 32 is made of a conductive material and two ends of the vent pipe 32 are inserted into the suction nozzle 2 and the atomization core 4, respectively, an airflow passage is surrounded in the inside of the vent pipe 32 for flowing the airflow to the suction nozzle 2, and the e-liquid storage cavity 33 for storing the e-liquid is formed between the vent pipe 32 and the e-liquid reservoir 31.

The connecting member 34 is fixed at one end of the e-liquid reservoir 31 for connection to the atomization core 4, and the atomization core 4 is detachably connected to the connecting member 34. In the present embodiment, the detachable connection between the connecting member 34 and the atomization core 4 is a threaded connecting structure, the connecting member 34 is provided with an internal thread at a position to be connected to the atomization core 4, an outer face of the atomization core 4 is provided with an external thread matched with the internal thread. If other detachable connecting structures are taken between the atomization core 4 and the connecting member 34, other connecting structures may be provided on the connecting member 34, such as a snap-on elastic piece having elasticity in a snap connection, and in the present embodiment, the threaded connecting connection is taken for an example, and it is not limited to the threaded connecting connection.

As shown in FIG. 1, an outer end surface of the suction nozzle 2 is provided with a second conductor 21, and the second conductor 21 is electrically connected to the vent pipe 32. The suction nozzle 2 is fixed at one end of the e-liquid reservoir 31, the end of the e-liquid reservoir 31 is opposite to the battery assembly 5. Two ends of the vent pipe 32 are inserted into the suction nozzle 2 and the atomization core 4, respectively, and an air outlet (not labeled in figures) communicating with the vent pipe 32 is provided in the suction nozzle 2, the smoke generated by the atomization core 4 sequentially passes through the vent pipe 32 and the air outlet for the user to inhale.

Preferably, the outer end surface of the suction nozzle 2 is spherical so that the suction nozzle 2 is beautiful and the outer end surface of the nozzle 2 is easy to clean dirt. In addition, since the suction nozzle 2 having a spherical outer end surface which is structurally stable, it is possible to prevent the suction nozzle 2 from being easily deformed and falling down when a hard object such as teeth in the mouth is held on the suction nozzle 2.

Preferably, the suction nozzle 2 is a metallic material and can prevent the suction nozzle 2 from being easily peeled off when the hard object such as the teeth of the mouth is held on the e-liquid reservoir 31. In this case, the metal may be copper, iron or steel, and is not particularly limited thereto.

The embodiment of the atomization core 4 of the atomization module 1 will be described in detail with reference to the embodiment shown in FIGS. 1, 2, 4, 5 and 6.

As shown in FIG. 4, the atomization core 4 mainly comprises an atomization seat 41, a heating wire assembly 42, a fixing block 43, an atomization cover 44, an electrode holder 45, and an atomization electrode assembly 46.

The atomization seat 41 is generally a hollow structure in which an air passage 32 is formed, the air passage 32 is communicated with the e-liquid cup assembly 3, and a side wall of the atomization seat 41 is provided with an e-liquid entering channel communicating with the e-liquid storage cavity 33. The heating wire assembly 42 is provided in the smoke passage and the smoke generated by the atomization of the heating wire assembly 42 is discharged from the vent pipe 32 through the smoke passage.

Specifically, the atomization seat 41 is made of a conductive material which comprises a first base body 411 and a second base body 412 connected to each other, and the first base body 411 and the second base body 412 are in interference fit. The first base body 411 is defined on one side of the atomization seat 41 close to the suction nozzle 2 and is in a plug connection with the vent pipe 32. The second base body 412 is defined on the other side of the atomization seat 41 close to the battery assembly 5 and is detachably connected to the battery assembly 5.

As shown in FIG. 5, an outer wall of the first base body 411 is provided with an external thread structure detachably connected to the connecting member 34 of the e-liquid cup assembly 3. The first base body 411 is provided with an installing groove 413 at one end thereof close to the suction nozzle 2, and the heating wire assembly 42 is provided in the installing groove 413. In the present embodiment, the installing groove 413 comprises two, two installing grooves 413 are spaced apart from each other and are in communication with the e-liquid storage cavity 33 of the e-liquid cup assembly 3, the e-liquid adsorbent 421 of the heating wire assembly 42 is installed and fixed at a bottom of the installing grooves 413 and extends into the e-liquid storage cavity 33. Further, the two installing grooves 413 constitute the e-liquid entering channel inside the atomization seat 41, and the e-liquid in the e-liquid storage cavity 33 is absorbed by the e-liquid adsorbent 421 through the e-liquid entering channel.

The heating wire assembly 42 comprises the e-liquid adsorbent 421 and an heating wire 422 (as shown in FIG. 4), the e-liquid adsorbent 421 is made of an e-liquid absorbing material which is fixed to the bottom of the installing grooves 413 and two ends of the e-liquid adsorbents 421 extend into the e-liquid storage cavity 33, the heating wire 422 is wound around the e-liquid adsorbent 421 and electrically connected to the battery assembly. Since the heating wire assembly 42 is a conventional means of the prior art, a specific structure thereof will not be described here.

The fixing block 43 is substantially a ring-shaped structure which is embedded and fixed to an inner wall of one end of the first base body 411 close to the suction nozzle 2. As shown in FIG. 4, an elastic conductive arm 431 extending to the smoke passage of the atomization 4 is fixed on the fixing block 43. The vent pipe 32 is inserted in the smoke passage and a side wall of the elastic conductive arm 431 and a side wall of the vent pipe 32 are resiliently abutted against and electrically connected to each other.

Further, the elastic conductive arm 431 is electrically connected to the atomization electrode assembly 46, as shown in FIG. 6, the elastic conductive arm 431 is provided with a perforation 432 provided with an electric wire (not shown in figures), one end of the electric wire is connected to the elastic conductive arm 431 and the other end of the electric wire is connected to the atomization electrode assembly 46 so that the elastic conductive arm 431 is electrically connected to the atomization electrode assembly 46. Since the elastic conductive arm 431 is electrically connected to the vent pipe 32 and the suction nozzle 2, respectively, thereby achieving that the atomization electrode assembly 46 is electrically connected to the vent pipe 32 and the suction nozzle 2.

The atomization cover 44 is provided with a receiving chamber (not labeled in figures) in an axial direction, and one end of the atomization cover 44 opposite to the suction nozzle 2 is sleeved on the first base body 411 and abuts against the heating wire assembly 42 to prevent the heating wire assembly 42 from being rocked. The vent pipe 32 (shown in FIG. 3) is inserted from one end of the atomization cover 44 close to the suction nozzle 2 and is in interference with a side wall of the receiving chamber.

Further, in order to prevent the e-liquid leaking from a connection of the vent pipe 32 with the atomization cover 44, a sealing ring (not labeled in figures) set on the vent pipe 32 may be provided in the receiving chamber of the atomization cover 44, an outer peripheral surface of the seal ring is elastically abutted against a side wall of the receiving chamber, and an inner peripheral surface of the seal ring is elastically abutted against an outer peripheral wall of the vent pipe 32 to achieve as a sealing function.

As shown in FIG. 5, the second base body 412 is generally a hollow cylindrical structure which is in a plug connection with and is in an interference fit with the first base body 411, a side of the second base body 412 close to the battery assembly 5 is detachably connected to the battery assembly 5. In the present embodiment, the detachable connection between the second base body 412 and the battery assembly 5 is a screw connection structure, and one side of the second base body 412 close to the battery assembly 5 is provided with an external thread structure, a connection position of the battery assembly 5 which is configured for being connected with the second base body 412 is provided with an internal thread structure adapted to the external thread structure. If other detachable connection structures are taken between the battery assembly 5 and the second base body 412, other detachable connection structures may be provided on the second base body 412, for example, a snap-in elastic piece having an elasticity in a snap connection, a buckle or a slot in an interlocking structure, etc. In this embodiment, only a screw connection structure is described as an example, and is not limited to a screw connection structure.

The electrode holder 45 is made of an insulating material and is fixed in the second base body 412. The electrode holder 45 is provided with a vent hole 451 communicating with the vent pipe 32. As shown in FIG. 4, the atomization electrode assembly 46 is fixed inside the electrode holder 45.

The atomization electrode assembly 46 is fixed on the electrode holder 45, and one end of the atomization electrode assembly 46 is electrically connected to the elastic conductive arm 431 through an electric wire, and the other end of the atomization electrode assembly 46 is electrically connected to the battery assembly 5. Specifically, the atomization electrode assembly 46 comprises a third electrode 461 and a fourth electrode 462, and the third electrode 461 and the fourth electrode 462 are both spring electrodes. The third electrode 461 is electrically connected to the elastic conductive arm 431 through an electric wire, and two ends of the heating wire 422 are electrically connected to the fourth electrode 462 and the atomization seat 41, respectively.

As shown in FIG. 4, the spring electrode comprises a protective sheath (not labeled in figures), and the protective sheath is provided with a groove (not labeled in figures), a spring and an electrical connector (all are not labeled on the drawings) are slidably connected to the groove. Specifically, one end of the spring is fixed to the bottom of the recess and the other end of the spring is fixedly connected to the electrical connector so that the electrical connector is slidably connected to the groove. When the atomization core 4 is connected to the battery assembly 5, the electrical connector is resiliently abutted against the battery electrode assembly 57 of the battery assembly 5 to electrically connect to the battery assembly 5.

Preferably, a non-slip knurled structure (not shown) may be provided on an outer surface of the second base body 412 in order to facilitate the user to rotate the atomization seat 41 when the atomization core 4 needs to be removed.

It is preferable that an outer surface of the atomization seat 41 is provided with an air inlet hole 414 which communicates with the vent pipe 32. In this embodiment, the air inlet hole 414 successively passes through an outer wall of the second base body 412 and an outer wall of the first base body 411, and is communicated with the vent pipe 32 and the vent hole 451 on the electrode holder 45.

In the present embodiment, the atomization core 4 mainly comprises an atomization seat 41, an heating wire assembly 42, a fixing block 43, an atomization cover 44, an electrode holder 45, and an atomization electrode assembly 46. It is to be understood that the atomization core 4 of the present application is not limited to the above-described structure, but may be other structures as long as it can atomize the smoke e-liquid.

A specific structure of the battery assembly 5 will be described in detail with reference to the embodiment shown in FIGS. 1, 2 and 3.

As shown in FIG. 3, the battery assembly 5 comprises the first conductor 51, the battery 52, the control module 53, the airflow sensor 54, the connector 55, the end cap 56, and a battery electrode assembly 57.

The first conductor 51 is generally a hollow cylindrical structure, the battery 52 is received and fixed in the first conductor 51, the battery 52 is mainly configured for supplying power needed by work to the atomization core 4 and the control module 53. In the present embodiment, the first conductive sleeve is electrically connected to the power source, i.e., the first conductive sleeve is electrically connected to a positive electrode of the battery 52. Certainly, the power source 22 may be realized by a high-energy capacitor or the like, and is not particularly limited thereto.

The end cap 25 is fixed at one end of the first conductor 51 opposite to the suction nozzle 2, an air hole (not labeled in figures) for the airflow to flow is defined on the end cap 25. Preferably, the end cap 25 is made of a light-transmissive material.

The connector 55 is made of a conductive material and is electrically connected to the atomization seat 41 of the atomization core 4. The connector 55 is connected to one end of the first conductor 51 close to the atomization core 4, and the connector 55 is detachably connected to the atomization seat 41, and an inner wall of the connector 55 is provided with an internal thread structure for detachably connecting to the atomization seat 41.

The battery electrode assembly 57 is fixed to the connector 55 and is electrically connected to the atomization electrode assembly 46 of the atomization core 4. Specifically, the battery electrode assembly 57 comprises a first electrode 571, a first insulating sleeve 572, a second electrode 573, and a second insulating sleeve 574 which are sequentially connected from the inside to the outside. The third electrode 461 of the atomization electrode assembly 46 is elastically abutted against and electrically connected to the second electrode 573, and the fourth electrode 462 is elastically abutted and electrically connected to the first electrode 571.

The control module 53 and the airflow sensor 54 are fixed in the first conductor 51, respectively, the control module 53 is electrically connected to the airflow sensor 54 and the battery 52, respectively. The airflow sensor 54 is mainly used for detecting an airflow signal and generating a pulse signal, the control module 53 is mainly used for receiving the signal from the airflow sensor 54 and receiving the trigger signal of the first conductor 51 and second conductor 21, and the control module 53 controls the battery 52 to supply power to the atomization core 4 to atomize the e-liquid according to the trigger signal and the pulse signal.

During an operation of the electronic cigarette, the second conductor 21 of the suction nozzle 2 is electrically connected to the elastic conductive arm 431 through the vent pipe 32, and the elastic conductive arm 431 is electrically connected to the third electrode 461 of the atomization electrode assembly 46 through the electric wire, and the third electrode 461 is elastically abutted against and electrically connected to the second electrode 573, the second electrode 573 is electrically connected to the control module 53, while the control module 53 is electrically connected to the first conductor 51, and when the human body skin contacts the first conductor and the second conductor 21 respectively, the above-described structure constitutes an electrical connection circuit.

Further, one end of the heating wire 422 of the heating wire assembly 42 is electrically connected to the atomization seat 41, the atomization seat 41 is screwed and electrically connected to the connector 55, and the connector 55 is electrically connected to the control module 53, and the control module 53 is electrically connected to the airflow sensor 54 by an electric wire, the airflow sensor 54 is electrically connected to the battery 52 through an electric wire, the battery 52 is connected to the first electrode 571 of the battery electrode assembly 57 through an electric wire, and the first electrode 571 is elastically abutted against and electrically connected to the fourth electrode 462 of the atomization electrode assembly 46, and the fourth electrode 462 is electrically connected to the other end of the heating wire 422 to form an electrical connection circuit.

Specifically, when the first conductor 51 and the second conductor 21 are contacted simultaneously through user's skin, the user's skin enables the first conductor 51 and the second conductor 21 to be conducted; the control module receives the conduction signal produced by that the first conductor 51 and the second conductor 21 are conducted, and detects the smoking signal generated by the airflow sensor 54, the control module 53 controls the battery 52 to supply the electrical power to the atomization core 4 to atomize e-liquid according to the conduction signal and the smoking signal.

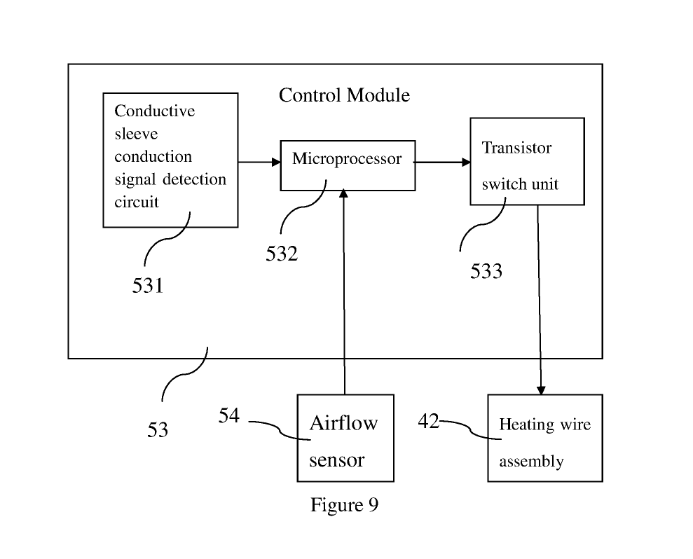

As shown in FIG. 9, the control module 53 comprises a conductive sleeve conduction signal detection circuit 531, a microprocessor 532, and a transistor switch unit 533, the transistor switch unit 533 is electrically connected to the microprocessor 532 and the heater wire assembly 42, respectively.

The microprocessor 532 in the control module 53 may be set with a heating time parameter after conduction, for example, in the microprocessor 532, heating time is 2 to 10 ms after receiving the conduction signal, and this time period is set for a main consideration of electrical contact problems, smokers do not have to keep pressing the two conductors, but only need to press instantly at the beginning to generate an instantaneous conduction signal, even without a conduction signal during the time period, a heating time can be maintained, it can overcome a problem that the heating atomization is immediately turned off after being conducted by the human body as a release of the user, so as to facilitate a use of the user.

The conductive sleeve conduction signal detection circuit 531 is configured for detecting whether or not the first conductive sleeve and the second conductor 21 are conducted, and transmitting a trigger signal generated after conduction to the microprocessor 532, meanwhile, the airflow sensor 54 detects an airflow signal and generates a pulse signal. The microprocessor 532 controls an on/off state of the transistor switch unit 533 in accordance with the trigger signal and the pulse signal to control whether the atomization core 4 atomizes the e-liquid.

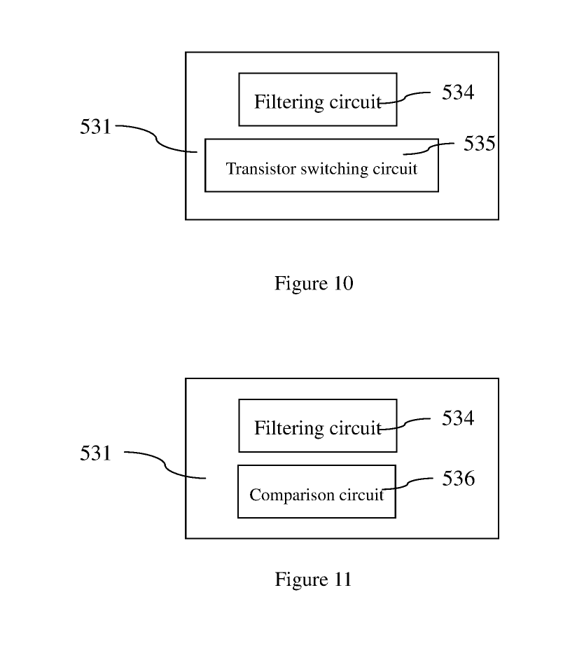

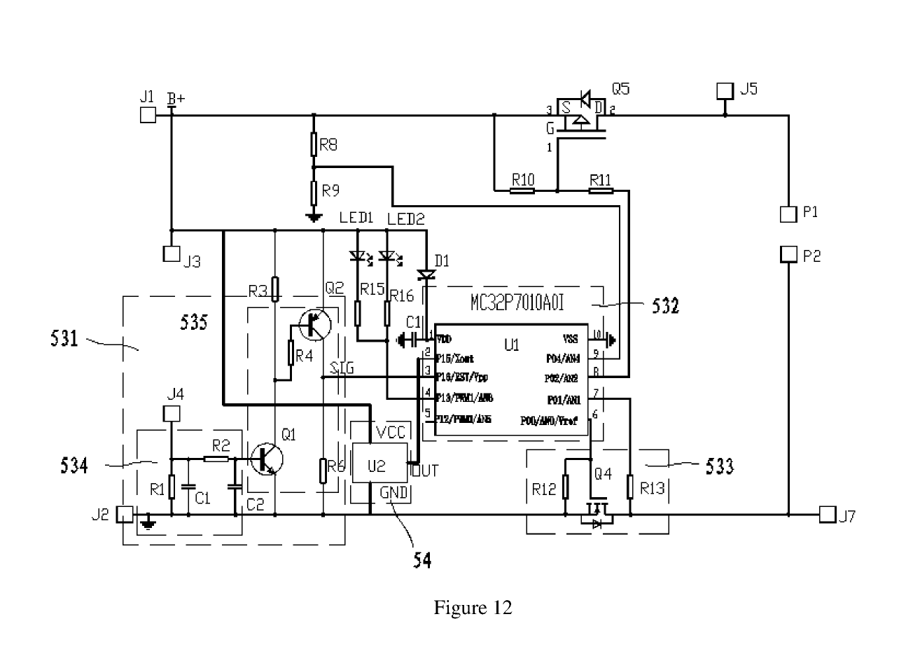

FIGS. 10 and 12 show a schematic structural view of the first control module 53 of the present embodiment, the conductive sleeve conduction signal detection circuit 531 comprises a filter circuit 534 and a transistor switching circuit 535. The filter circuit 534 is electrically connected to the second conductor 21 and is configured for filtering an input conduction signal; the transistor switching circuit 535 is electrically connected an output end of the filter circuit 534, the transistor switching circuit 535 is configured for receiving a sensing signal after filtering and outputting a switching signal configured as a trigger signal to the microprocessor 532.

Specifically, the filter circuit 534 comprises a first resistor R1, a second resistor R2, a first capacitor C1 and a second capacitor C2, a common terminal of the first resistor R1, the first capacitor C1 and the second capacitor C2 is grounded, the other end of the first resistor R1, the other end of the first capacitor C1 and one end of the second resistor R2 are electrically connected to the second conductor 21, the other end of the second resistor R2 and the other end of the second capacitor C2 are connected to the transistor switching circuit 535.

The transistor switching circuit 535 comprises a first transistor Q1, a second transistor Q2, a third resistor R3, a fourth resistor R4 and a fifth resistor R6; a base of the first transistor Q1 is connected with the filter circuit, an emitter of the first transistor Q1 is grounded, a collector of the first transistor Q1 is connected to one end of the third resistor R3 and one end of the fourth resistor R4, the other end of the third resistor R3 is electrically connected to the power source, and the other end of the fourth resistor R4 is connected to a base of the second transistor Q2, an emitter of the second transistor Q2 is electrically connected to the power source, a collector of the second transistor Q2 is electrically connected to one end of the fifth resistor R6 and the microprocessor 532, and the other end of the fifth resistor R6 is grounded.

The microprocessor 532 is applied as a microcontroller U1 whose type is MC32P7010A0I, the transistor switch unit 533 comprises a field effect transistor Q4, the heating wire 422 is connected between P1 and P2, J3 is electrically connected to the first conductor 51 and electrically connected to the positive electrode of the battery 52, J4 is electrically connected to the second conductor 21. During operation, a portion of the human body contacts the second conductor 21, and the other portion of the human body contacts the first conductor 51. A specific working principle of the circuit is as follows: when the human body is not in contact, a collector of the transistor Q2 outputs a low level, that is, a signal SIG=logic 0; when someone contacts the first conductor 51 and the second conductor 21, a signal is firstly filtered by the filter circuit 534, then flows through the transistor Q1 to turn on the transistor Q1, and then the transistor Q2 is turned on, so that the signal SIG are changed into a high level, that is, the signal SIG=logic 1. When the microcontroller U1 detects a signal changed from the low level to the high level, it is known that someone wants to smoke, the microcontroller U1 waits for a signal of the airflow sensor U2 until the airflow sensor U2 has an activated high level, and the microcontroller U1 controls the field effect transistor Q4 to turn on, that is, an effective high level of the airflow sensor U2, and the signal SIG=logic 1 (i.e., the first conductor 51 and the second conductor 51 are brought into contact), the atomization assembly can operate.

Through the above circuit, we have realized that when the human body contacts the first conductor 51 and the second conductor 51, and the airflow sensor 54 also has an effective smoking signal, the electronic cigarette can work properly, avoiding that the situation of the electronic cigarette automatic work takes place in the packaging and transportation process. Due to the packaging or sealing of an electronic cigarette may cause air pressures on both sides of the airflow sensor 54 is different, resulting in the electronic cigarette automatic work; or in the transportation process, such as aircraft, automatic operation of the electronic cigarette may be caused by changes in air pressure, and the electronic cigarette automatic operation for a long time may cause a risk of fire and explosion, and affecting user experience effect.

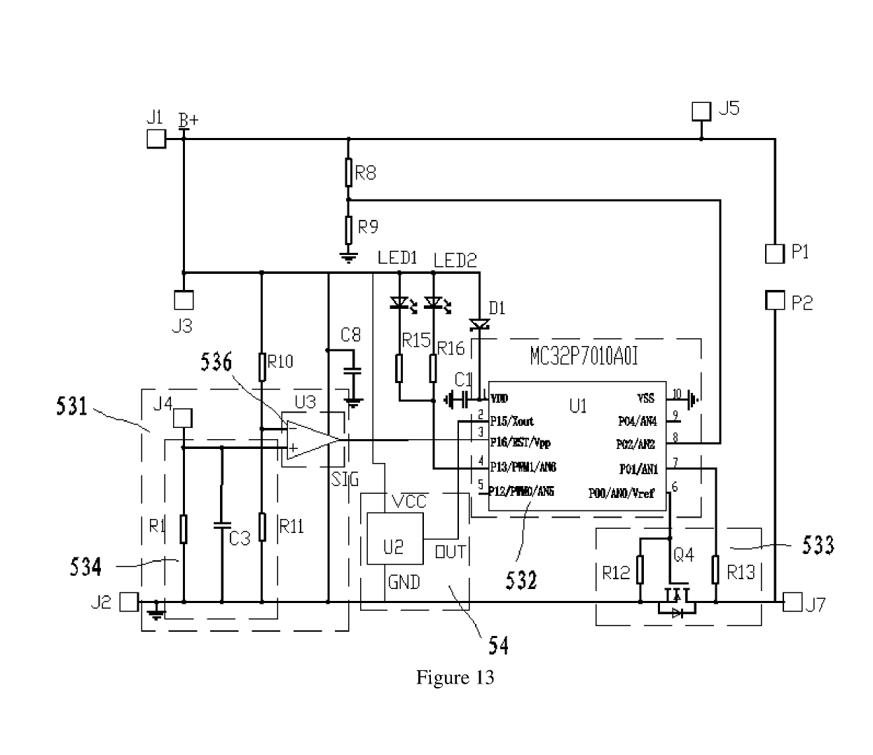

FIGS. 11 and 13 show a schematic structural view of the second control module 53 of the present embodiment, which differs from the first control module 53 in that the structure of the conductive sleeve conduction signal detection circuit 531 is different.

As shown in FIG. 11, the conductive sleeve conduction signal detection circuit 531 comprises a filter circuit 534 and a comparison circuit 236. The filter circuit 534 is electrically connected to the second conductor 21 and is used for filtering the input signal; The comparison circuit 236 is electrically connected to an output terminal of the filter circuit 534, the comparison circuit 236 comprises a comparator U3, a resistor R10 and a resistor R11, a reverse input terminal of the comparator U3 is electrically connected to a common terminal of the resistor R10 and the resistor R11, the resistor R10 and the resistor R11 are used to provide a divided voltage as a voltage reference to the reverse input terminal of the comparator U3; an output terminal of the comparator U3 is electrically connected to a pin 3 of the microprocessor 532, the other end of the resistor R10 is electrically connected to the first conductor 51 and the positive electrode of the battery, the other end of the resistor R11 is grounded, and a positive input terminal of the comparator U3 receives a filtered conductive signal and compares the filtered conductive signal with a reference voltage signal of the reverse input terminal, and an output terminal of the comparator U3 circuit outputs a comparison result signal as a trigger signal to the microprocessor 532.

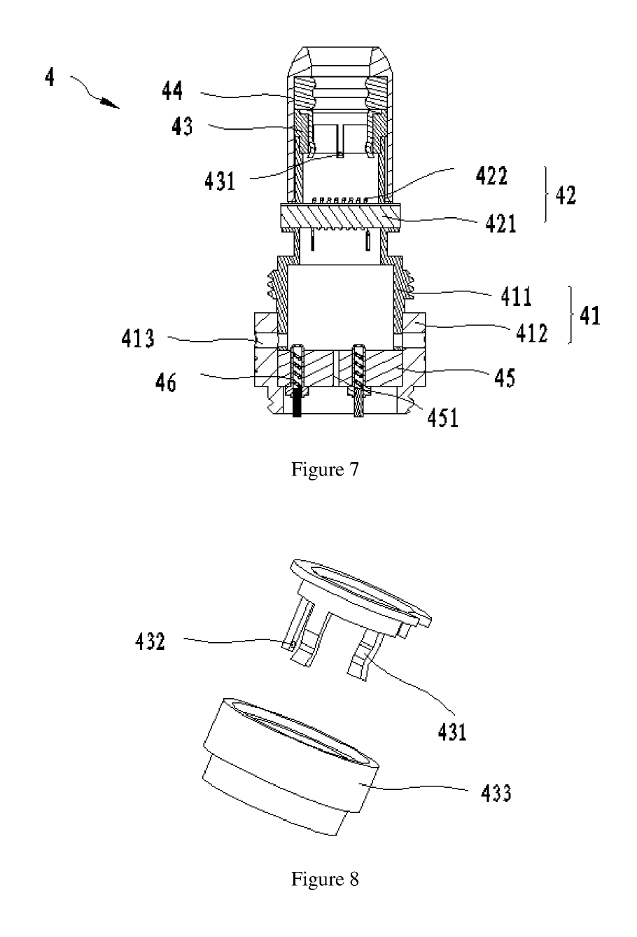

FIGS. 7 and 8 show an electronic cigarette provided in the second embodiment of the present invention, the second embodiment differs from the first embodiment in that the structure of the atomization core 4 is different.

As shown in FIG. 7, the fixing block 43 is substantially a ring-shaped structure which is embedded and fixed to an inner side wall of the first base body 411 close to one end of the suction nozzle 2. As shown in FIG. 8, the fixing block 43 is a silicone member, an elastic piece (not labeled in figures) is defined on the fixing block 3, the elastic piece has at least one elastic conductive arm 431 extending into the smoke passage of the atomization seat 41, the vent pipe 32 is inserted in the smoke passage, and the elastic conductive arm 431 is resiliently abutted against and electrically connected to a sidewall of the vent pipe 32.

The present embodiment arranges the fixing block 43 and the elastic piece structure independently from each other, and is actually more reasonable in practice, and the elastic piece structure has a plurality of elastic conductive arms 431, and by increasing the number of the elastic conductive arms 431, stability of electrical connections between the elastic conductive arms 431 and the vent pipe 32 is increased to prevent a contact between the elastic conductive arms 431 and the vent pipe 32 from being poor.

Further, one elastic conductive arm 431 is electrically connected to the atomization electrode assembly 46, and as shown in FIG. 8, the elastic conductive arm 431 is provided with a perforation 432 through which an electric wire (not labeled in figures) passes, one end of the electric wire is connected to the elastic conductive arm 431 and the other end of the electric wire is connected to the atomization electrode assembly 46 so that the elastic conductive arm 431 is electrically connected to the atomization electrode assembly 46. Since the elastic conductive arm 431 is electrically connected to the vent pipe 32 and the suction nozzle 2, then the atomization electrode assembly 46 is electrically connected to the vent pipe 32 and the suction nozzle 2. It is to be understood that the above-described connection between the atomization assembly 1 and the battery assembly 2 is a detachably connection, and certainly, the structure of the battery assembly 2 and the atomization assembly 1 may be an in-detachable and integrated structure, namely, the electronic cigarette may be a disposable electronic cigarette, or other electronic cigarettes capable of repeatedly adding e-liquid to be repeatedly used, and the structure thereof is not particularly limited thereto.

The present invention further provides an atomization control method of an electronic cigarette, the electronic cigarette is the above electronic cigarette, the atomization control method of an electronic cigarette comprises following steps:

S1. conducting the first conductor 51 and the second conductor 21 by user's skin when the user contacts the first conductive sleeve and the second conductor 21 through the skin simultaneously, so as to generate a conduction signal to the control module 53;

S2. controlling the power source to supply the electrical power to the heating wire assembly 42 to atomize e-liquid when the control module 53 receives the conduction signal and a smoking signal, the conduction signal is generated by that the first conductor 51 and the second conductor 21 are conducted.

In general, the electronic cigarette and an atomization control method thereof of the present invention has following advantageous effects:

(1) The first conductor is provided on the outer peripheral surface of the battery assembly, and the outer peripheral surface of the suction nozzle is provided with a second conductor, when smoking, the user directly holds on the first conductive sleeve by fingers, a mouth is held on the suction nozzle, so as to realize contacts between the conductive sleeves at different positions on the outer surface of the electronic cigarette and the user skin at the same time, and to transmit the conduction signal to the control module so that the control module controls the power source to supply the heating wire assembly to atomize the e-liquid and starts the electronic cigarette working.

Therefore, the present invention can reduce a probability that the electronic cigarette is mistakenly triggered, effectively solves the problem in the prior art that the electronic cigarette works when the key switch, the touch switch or the airflow sensor is often mistakenly triggered, resulting in a change of deterioration of taste of the e-liquid of the electronic cigarette caused by repeated oxidization and, a short electronic cigarette life, low safety and other issues. Moreover, by providing the structure of the present invention, it is possible to reduce conditions of the packaging apparatus for transporting the electronic cigarette, thus to facilitate the transportation and the transportation is more secure. In addition, during a using process, the structure meets user requirements that the user can smoke when the fingers are held on the battery assembly and the mouth is held on the suction nozzle, it is more user-friendly, improves user experience, so as to avoid a complex using problem that the touch keys or switches are needed in the prior art, and to avoid the problem that the airflow sensor cannot detect the smoke signal when the smoking force is small, the e-liquid enters the battery assembly from the airflow channel, and other problems.

(2) Since the atomization assembly is detachably connected to the battery assembly, it is convenient for the user to replace the atomization assembly after that the atomization assembly is damaged.

While the embodiments of the present application are described with reference to the accompanying drawings above, the present application is not limited to the above-mentioned specific implementations. In fact, the above-mentioned specific implementations are intended to be exemplary not to be limiting. In the inspiration of the present application, those ordinary skills in the art can also make many modifications without breaking away from the subject of the present application and the protection scope of the claims. All these modifications belong to the protection of the present application.

1. An electronic cigarette comprising an atomization assembly (1) and a battery assembly (5), wherein a heating wire assembly (42) is defined in the atomization assembly (1), the battery assembly (5) is provided with a power source for supplying electrical power to the heating wire assembly (42) so that the atomization assembly (1) atomizes e-liquid to form smoke; wherein the electronic cigarette further comprises a control module (53) electrically connected to the power source and the heating wire assembly (42), respectively, and an airflow sensor (54) configured for transmitting a smoking signal to the control module (53) when the airflow sensor (54) detects that a user is smoking; a first conductor (51) is provided on an outer peripheral surface of the battery assembly (5); a suction nozzle (2) for the user to smoke is provided on an end of the atomization assembly (1) opposite to the battery assembly (5); and a second conductor (21) is provided on an outer peripheral surface of the suction nozzle (2); wherein one conductor of the first conductor (51) and the second conductor (21) is electrically connected to the control module (53) and the other conductor is electrically connected to the power source; and
wherein the first conductor (51) and the second conductor (21) are contacted and then conducted by the user's skin while the user is smoking, the control module (53) is configured for controlling the power source to supply the electrical power to the heating wire assembly (42) to atomize the e-liquid when the control module (53) detects the smoking signal and a conduction signal generated by that the first conductor (51) and the second conductor (21) are conducted.

2. The electronic cigarette according to claim 1, wherein the atomization assembly (1) comprises an e-liquid cup assembly (3), the e-liquid cup assembly (3) comprises an e-liquid reservoir (31) and a vent pipe (32), the vent pipe (32) is inserted in the e-liquid reservoir (31) and configured for discharging smoke atomized by the atomization assembly (1) from the suction nozzle (2); one end of the e-liquid cup assembly (3) is provided with the suction nozzle (2) made of a conductive material, an outer surface of the suction nozzle (2) is configured as the second conductor (21), the other end of the e-liquid cup assembly (3) is provided with an atomization core (4) for atomizing the e-liquid, at least one end of the atomization core (4) is inserted into the e-liquid cup assembly (3); and
wherein one end of the atomization core (4) is connected to the vent pipe (32), the other end of the atomization core (4) is connected with the battery assembly (5), the control module (53) is defined in the battery assembly (5), two ends of the vent pipe (32) are electrically connected to the suction nozzle (2) and the atomization core (4), respectively, the suction nozzle (2) is electrically connected to the control module (53) via the vent pipe (32) and the atomization core (4).

3. The electronic cigarette according to claim 2, wherein a smoke passage and an elastic conductive arm (431) are defined in the atomization core (4), the smoke passage is communicated with the vent pipe (32), the elastic conductive arm (431) extends into the smoke passage, the elastic conductive arm (431) is electrically connected to the control module (53), the vent pipe (32) is inserted into the smoke passage, the elastic conductive arm (431) is abutted against a side wall of the vent pipe (32).

4. The electronic cigarette according to claim 3, wherein the atomization core (4) further comprises a hollow atomization seat (41); the smoke passage is defined in the atomization seat (41), a side wall of the atomization seat (41) is provided with an e-liquid entering channel communicated with an e-liquid storage cavity (33) in the e-liquid cup assembly (3), the heating wire assembly (42) is defined on the smoke passage; an end of the atomization seat (41) is connected with an atomization electrode assembly (46) configured for being electrically connected to the battery assembly (5); and
wherein the heating wire assembly (42) comprises an e-liquid adsorbent (421) for adsorbing e-liquid in the e-liquid storage cavity (33) from the e-liquid entering channel, and a heating wire (422) for atomizing the e-liquid on the e-liquid adsorbent (421); the heating wire (422) is electrically connected to the atomization electrode assembly (46).

5. The electronic cigarette according to claim 1, wherein the battery assembly (5) comprises a first conductive sleeve configured as the first conductor (51), the first conductive sleeve receives a battery (52) configured as the power source, one end of the first conductive sleeve is provided with a connector (55) for being detachably connected the atomization assembly (1), the other end of the first conductive sleeve is provided with an end cap (56) configured for covering the first conductive sleeve.

6. The electronic cigarette according to claim 1, wherein the battery assembly (5) comprises a battery case, the first conductor (51) is a first conductive sleeve made of a conductive paper, the first conductive sleeve is sleeved on the battery case and covers an entire outer peripheral surface of the battery case; the battery case receives a battery (52) configured as the power source, one end of the battery case is provided with a connector (55) for being detachably connected the atomization assembly (1), the other end of the battery case is provided with an end cap (25) configured for covering the first conductive sleeve.

7. The electronic cigarette according to claim 6, wherein an outer peripheral surface of the atomization assembly (1) is provided with an insulating paper sleeve defined coaxially with the first conductor (51), the insulating paper sleeve is located between the first conductor (51) and the second conductor (21).

8. The electronic cigarette according to claim 1, wherein the battery assembly (5) and the atomization assembly (1) are detachably connected, and the detachable connection between the battery assembly (5) and the atomization assembly (1) is a threaded connection structure or an interlocking connection structure.

9. The electronic cigarette according to claim 1, wherein the control module (53) comprises a conductive sleeve conduction signal detection circuit (531), a microprocessor (532), a transistor switch unit (533), the transistor switch unit (533) is electrically connected to the microprocessor (532) and the heating wire assembly (42); and
wherein the conductive sleeve conduction signal detection circuit (531) is configured for detecting whether or not the first conductor (51) and the second conductor (21) are conducted, and transmitting a trigger signal generated after the conduction to the microprocessor (532), the microprocessor (532) is configured for controlling an on/off state of the transistor switch unit (533) in accordance with the trigger signal and the smoking signal which is outputted by the airflow sensor (533) to control whether the atomization assembly (1) atomizes the e-liquid.

10. The electronic cigarette according to claim 9, wherein the conductive sleeve conduction signal detection circuit (531) comprises a filter circuit (534) electrically connected to the second conductor (21) for filtering an inputted conduction signal, and a transistor switching circuit (535) electrically connected an output end of the filter circuit (534), the transistor switching circuit (535) is configured to receive the conduction signal after filtering and output a switching signal to the microprocessor (532).

11. The electronic cigarette according to claim 10, wherein the filter circuit (534) comprises a first resistor (R1), a second resistor (R2), a first capacitor (C1) and a second capacitor (C2), a common terminal of the first resistor (R1), the first capacitor (C1) and the second capacitor (C2) is grounded, the other end of the first resistor (R1), the other end of the first capacitor (C1) and one end of the second resistor (R2) are electrically connected to the second conductor (21), the other end of the second resistor (R2) and the other end of the second capacitor (C2) are connected to the transistor switching circuit (535).

12. The electronic cigarette according to claim 10, wherein the transistor switching circuit (535) comprises a first transistor (Q1), a second transistor (Q2), a third resistor (R3), a fourth resistor (R4) and a fifth resistor (R6); a base of the first transistor (Q1) is connected with the filter circuit, an emitter of the first transistor (Q1) is grounded, a collector of the first transistor (Q1) is connected to one end of the third resistor (R3) and one end of the fourth resistor (R4), the other end of the third resistor (R3) is electrically connected to the power source, and the other end of the fourth resistor (R4) is connected to a base of the second transistor (Q2), an emitter of the second transistor (Q2) is electrically connected to the power source, a collector of the second transistor (Q2) is electrically connected to one end of the fifth resistor (R6) and the microprocessor (532), and the other end of the fifth resistor (R6) is grounded.

13. The electronic cigarette according to claim 9, wherein the conductive sleeve conduction signal detection circuit (531) comprises a filter circuit (534) electrically connected to the second conductor (21) for filtering an inputted conduction signal, and a comparison circuit (536) electrically connected to an output terminal of the filter circuit (534), a positive input terminal of the comparison circuit (236) receives the conduction signal after filtering and outputs a comparison result signal to the microprocessor (532).

14. The electronic cigarette according to claim 1, wherein the battery assembly (5) is provided with a battery (52) configured as the power source; one conductive sleeve selected from both the first conductor (51) and the second conductor (21) is electrically connected to a positive electrode of the battery (52).

15. The electronic cigarette according to claim 1, wherein the atomization assembly (1) and the battery assembly (5) are arranged coaxially.

16. An atomization control method of an electronic cigarette, the electronic cigarette comprises an atomization assembly (1) and a battery assembly (5), a heating wire assembly (42) is defined in the atomization assembly (1), the battery assembly (5) is provided with a power source for supplying electrical power to the heating wire assembly (42) so that the atomization assembly (1) atomizes e-liquid to form smoke; wherein the electronic cigarette further comprises a control module (53) electrically connected to the power source and the heating wire assembly (42) respectively, and an airflow sensor (54) configured for transmitting a smoking signal to the control module (53) when the airflow sensor (54) detects that a user is smoking; a first conductor (51) is provided on an outer peripheral surface of the battery assembly (5); a suction nozzle (2) for a user to smoke is provided on an end of the atomization assembly (1) opposite to the battery assembly (5); and a second conductor (21) is provided on an outer peripheral surface of the suction nozzle (2); the control method comprises following steps:
S1. conducting the first conductor (51) and the second conductor (21) with user's skin when the user contacts the first conductor (51) and the second conductor (21) with the skin simultaneously, so as to generate a conduction signal to the control module (53);
S2. controlling the power source to supply the electrical power to the heating wire assembly (42) to atomize e-liquid when the control module (53) receives the conduction signal and a smoking signal, the conduction signal is generated by that the first conductor (51) and the second conductor (21) are conducted.
